SEQUENCE LISTING (5 sequences)

SEQ ID NO: 1 - DNA, 20 bp, artificial sequence
  synthetic
tttttttttttttttttttt

SEQ ID NO: 2 - DNA, 20 bp, artificial sequence
  synthetic
aaaaaaaaaaaaaaaaaaaa

SEQ ID NO: 3 - DNA, 202 bp, artificial sequence
  synthetic
ttggtagcaaaaacgaactgggttcaatcttggctctgccacccattcctggacaaaatacttaactctctccatcttccaaatgtgacctacaccttacaggactgccaggagaattaaggaagcacatacacatggcctgacacacaagtgagtaaacaccttaaactatgtttagccacagaaccagagtggttggagg

SEQ ID NO: 4 - DNA, 44 bp, artificial sequence
  synthetic
ccctggaccctggaccctggaccctggccctggaccctggagaa

SEQ ID NO: 5 - DNA, 44 bp, artificial sequence
  synthetic
ttctccagggtccagggccagggtccagggtccagggtccaggg

(19) United States
(12) Patent Application Publication
(10) Pub. No.: US 2017/0058344 A1
(43) Pub. Date: Mar. 2, 2017

(54) Mapping protein bound regions on dsDNA by reaction of unblocked Thymidines with Osmium tetroxide 2,2'-bipyridine.

(71) Applicant: Anastassia Kanavarioti, El Dorado Hills, CA (US)

(72) Inventor: Anastassia Kanavarioti, El Dorado Hills, CA (US)

(21) Appl. No.: 15/253,393

(22) Filed: Aug. 31, 2016

Related U.S. Application Data
(60) Provisional application No. 62/212,072, filed on Aug. 31, 2015.

(51) Int. Cl. C12Q 1/68; G01N 27/447; G01N 33/487

(52) U.S. Cl. CPC C12Q 1/6874; G01N 33/48721; C12Q 1/6806; G01N 27/44791; G01N 27/4473

(57) ABSTRACT

Materials, methods, and systems for determining the position, i.e. the sequence within a double stranded nucleic acid, where a protein is bound are disclosed and described. Materials can include dsDNA, dsRNA, and dsDNA-mimics. Materials are first reacted with Osmium tetroxide 2,2′-bipyridine to partially osmylate the nucleic acid (osmylated or labeled polymer) and monitored by HPLC or CE in order to stop the labeling before it reaches 50%. This preserves the double stranded structure of the nucleic acid and leaves the protein bound to it during the labeling. Labeling occurs randomly at the whole length of the polymer but leaves the region where the protein binds intact. Methods are provided to describe preparation of the osmylated polymers, their purification, and characterization. Labeled polymers may be subject to voltage-driven translocation via nanopores of appropriate width so that the polymer can traverse. There are two choices: Either use nanopores suitable for dsDNA, or use nanopores suitable for ssDNA and denature the labeled polymer before testing. Either way the translocation is monitored and reported as a current vs. time (i-t) profile. The current is stable, but fluctuates during the polymer's translocation in a manner that pinpoints the region where characteristically no osmylated base appears (less current reduction in i-t), while the majority of the trace will exhibit a pattern corresponding to the osmylated bases interspersed among the intact bases (more current reduction in i-t). The region where the protein is bound can be inferred by comparison of the i-t data of the two osmylated polymers (with or without bound protein).

This application claims the benefit of U.S. Provisional Application:
U.S. PTO No. 62/212,072 filed on Aug. 31, 2015 entitled “Mapping protein bound regions on dsDNA by reaction of unblocked Thymidines with Osmium tetroxide 2,2′-bipyridine”, by Dr. Anastassia Kanavarioti, inventor. The contents of the above are hereby incorporated by reference in its entirety into this application.

This application also claims the benefit of U.S. non provisional Application:
U.S. PTO Ser. No. 14/944,888 filed on Nov. 18, 2015 entitled “Labeled Nucleic Acids: A Surrogate for Nanopore-based Nucleic Acid Sequencing”, by Dr. Anastassia Kanavarioti, inventor. The contents of the above are hereby used as reference and additional evidence into this application.

Publications of the inventor relevant to this invention:
1. Kanavarioti A, Greenman K L, Hamalainen M, Jain A, Johns A M, Melville C R, Kemmish K, and Andregg W. Capillary electrophoretic separation-based approach to determine the labeling kinetics of oligodeoxynucleotides, Electrophoresis. 2012, 33(23):3529-3543. doi: 10.1002/elps.201200214.
2. Kanavarioti A. Osmylated DNA, a novel concept for sequencing DNA using nanopores. Nanotechnology. 2015, 26(13):134003. doi: 10.1088/0957-4484/26/13/134003.
3. Kanavarioti, A. “A non-traditional Approach to Whole Genome ultra-fast, inexpensive Nanopore-based Nucleic Acid Sequencing”, Austin J Proteomics Bioinform & Genomics. 2015, 2(2), 1012.
4. Henley R Y, Vazquez-Pagan A G, Johnson M, Kanavarioti A, Wanunu M. “Osmium-Based Pyrimidine Contrast Tags For Enhanced Nanopore-Based DNA Base Discrimination”, PLoS One. 2015, 10(12):e0142155. doi: 10.1371/journal.pone.0142155.
5. Ding, Y, Kanavarioti, A. “Single Pyrimidine Discrimination during Voltage-driven Translocation of Osmylated Oligodeoxynucleotides via the α-Hemolysin Nanopore”, Beilstein J Nanotechnol. 2016, 7:91-101. doi: 10.3762/bjnano.7.11.
6. Kanavarioti, A. “False Positives and false negatives measure less than 0.001% in labeling ssDNA with Osmium tetroxide 2,2′-bipyridine”, Beilstein J Nanotechnol. 2016, submitted.

Publications and Patents of others in the same fields of science as this invention:
7. Palecek E. Probing DNA structure with Osmium Tetroxide Complexes in Vitro. Methods in Enzymology 1992, 212, 139-55. Please note that under our conditions osmylation of the ribose is not detectable.
8. Beer, M. and Moudrianakis, E. N. Determination of Base Sequence in Nucleic Acids with the Electron Microscope: Visibility of a Marker. Proc. Natl. Acad. Sci. U.S.A. 1962, 48, 409-416.
9. Chang, C-H.; Beer, M.; Marzilli, L. G. Osmium-Labeled Polynucleotides. The reaction of osmium Tetroxide with Deoxyribonucleic Acid and Synthetic polynucleotides in the presence of tertiary Nitrogen Donor Ligands. Biochemistry 1977, 16, 33-38.
10. Chang C H, Beer M, Marzilli L G. Osmium-labeled polynucleotides. The reaction of osmium tetroxide with deoxyribonucleic acid and synthetic polynucleotides in the presence of tertiary nitrogen donor ligands. Biochemistry. 1977, 16: 33-8.
11. Palecek E, Vlk D, Vojtísková M, Boublíková P. Complex of osmium tetroxide with 1,10-phenanthroline binds covalently to double-stranded DNA. J Biomol. Struct. Dyn. 1995,13, 537-546.
12. Nomura, A., Okamoto, A. Reactivity of thymine doublet in single strand DNA with osmium reagent. Nucleic Acids Symp. Ser. 2008, 52, 433-4.
13. Dobi, A. L.; Matsumoto, K.; Santha, E.; vAgoston, D. Guanine specific chemical sequencing of DNA by osmium tetroxide. Nuc. Acids Res. 1994, 22, 4846-4847.
14. Kasianowicz, J. J. Introduction to ion channels and disease. Chem. Rev. 2012, 112, 6215-7.
15. Hague, F.; Li, J.; Wu, H. C.; Liang, X. J.; Guo, P. Solid-State and Biological Nanopore for Real-Time Sensing of Single Chemical and Sequencing of DNA. Nano Today 2013, 8, 56-74.
16. Maglia, G.; Heron, A. J.; Stoddart, D.; Japrung, D.; Bayley, H. Analysis of single nucleic acid molecules with protein nanopores. Methods Enzymol. 2010, 475, 591-623.
17. Wolna, A. H.; Fleming, A. M.; An, N.; He, L.; White, H. S. and Burrows, C. J. Electrical Current Signatures of DNA Base Modifications in Single Molecules Immobilized in the α-Hemolysin Ion Channel. Isr. J. Chem. 2013, 53, 417-430.
18. Mitchell, N.; Howorka, S. Chemical tags facilitate the sensing of individual DNA strands with nanopores. Angew. Chem. Int. Ed. Engl. 2008, 47, 5565-8. PMID: 18553329
19. Kumar, S.; Tao, C.; Chien, M.; Hellner, B.; Balijepalli, A.; Robertson, J. W. F.; Li, Z.; Russo, J. J.; Reiner, J. E.; Kasianowicz, J. J. and Ju, J. PEG-Labeled Nucleotides and Nanopore Detection for Single Molecule DNA Sequencing by Synthesis. Scientific Reports 2012, 2, 684.
20. Borsenberger, V.; Mitchell, N.; Howorka, S. Chemically labeled nucleotides and oligonucleotides encode DNA for sensing with nanopores. J. Am. Chem. Soc. 2009, 131, 7530-7531. doi: 10.1021/ja902004s.
21. Furey T. S. ChIP-seq and Beyond: new and improved methodologies to detect and characterize protein-DNA interactions Nat Rev Genet. 2012, 13, 840-852.
22. Mendenhall E M, Bernstein B E. DNA-protein interactions in high definition. Genome Biol. 2012, 13(1):139. doi: 10.1186/gb-2012-13-1-139.
23. Orenstein Y, Shamir R. Modeling protein-DNA binding via high-throughput in vitro technologies. Brief Funct Genomics. 2016, 1-10.
24. Glick Y, Orenstein Y, Chen D, Avrahami D, Zor T, Shamir R, Gerber D. Integrated microfluidic approach for quantitative high-throughput measurements of transcription factor binding affinities. Nucleic Acids Res. 2016, 44(6):e51. doi: 10.1093/nar/gkv1327.

| Application # | Filed: | Issued: | Title |
|---|---|---|---|
| 20150152495 | Nov. 26, 2014 | Jun. 4, 2015 | Compositions and Methods for Polynucleotide |
| | | | Sequencing |
| WO2013/041878 | Sep. 21, 2012 | Mar. 28, 2013 | Analysis of a Polymer comprising Polymer Units |
| US5795782A1, US6015714A1 | Mar. 17, 1995 | Aug. 18, 1998 | Characterization of individual polymer molecules |
| EP0815438B1, | | | based on monomer-interface interactions |
| Claimed IP of Oxford | | | Use of solid state nanopores for detecting labeled |
| Nanopore Technologies | | | ssDNA and dsDNA |
| 7,825,248 | Jan. 23, 2009 | Nov. 2, 2010 | Synthetic nanopores for DNA sequencing |
| 20030099951, 6,936,433 | Nov. 21, 2001 | May 29, 2003 | Methods and Devices for characterizing duplex |
| | | | nucleic acid molecules |
| 20150119259 | Apr. 8, 2013 | Apr. 30, 2015 | Nucleic acid sequencing by nanopore detection of |
| | | | Tag molecules |
| 20150037788 | Oct. 17, 2014 | Feb. 5, 2015 | DNA sequencing by nanopore using modified |
| | | | nucleotides |
| 20130264207 | Dec. 16, 2011 | Oct. 10, 2013 | DNA sequencing by synthesis using modified |
| | | | nucleotides and nanopore detection |
| 20120142006 | Dec. 28, 2011 | Jun. 7, 2012 | Massive parallel method for decoding DNA and |
| | | | RNA |
| US9005425B2 | Sep. 7, 2011 | Apr. 14, 2015 | Detection of Nucleic acid Lesions and adducts using |
| | | | nanopores |
| US5217863A | Dec. 26, 1991 | Jun. 8, 1993 | Detection of mutations in nucleic acids |

### TERMS

As used herein, and unless stated otherwise, each of the following terms shall have the definition set forth below.
Osmylation—The reaction of a nucleic acid to form a nucleic acid conjugate where the T-bases are T(OsBp), or where all the pyrimidines are osmylated, (T+C)OsBp. Intermediate levels of T- and C-osmylation are possible, only that due to selectivity T is practically completely osmylated before C is osmylated.
Osmylated—material that was subject to osmylation
A—Adenine;
C—Cytosine;
DNA—Deoxyribonucleic acid; unless specifically mentioned all bases are deoxynucleotides.
G—Guanine;
T—Thymidine
For the purposes of this document and the experiments described herein: T=dT, C=dC, A=dA, G=dG, i.e. all the nucleotides here are deoxynucleotides. To identify the ribonucleotides the terms rA, rU, rC and rG will be used herein. ss-single stranded
ds—double stranded
dsOligo—double stranded oligo, a hybrid from the two complementary oligos
nt—nucleotide
bp—base pair
PBS—phosphate buffer saline
wt—wild type
α-HL or α-Hemolysin—the alpha Hemolysin nanopore
“Nucleic acid” or polynucleotide shall mean any nucleic acid molecule, including, without limitation, DNA, RNA and hybrids thereof. The nucleic acid bases that form nucleic acid molecules can be the bases A, C, G, T, U, in the deoxy or the ribodeoxyform, as well as derivatives thereof that comprise the so called non-canonical or rare bases found mostly in tRNAs.
OsBp or bipy-Os04 or Osbipy—Osmium tetroxide 2,2′-bipyridine (see FIG. 1)
nanopore or channel—natural or solid-phase nanopores, channels, hybrids thereof, or massively parallel devices or instruments including them.
CE—Capillary Electrophoresis: Typical methods comprise an untreated fused-silica capillary (50 um ID×40 cm) with extended light path purchased from Agilent. Typical buffers are 50 mM phosphate pH 7 or 50 mM borate pH 9.2 with 20 kV, 25 kV or 30 kV.
HPLC—High Performance Liquid Chromatography: Typical methods comprise Ion-exchange with DNA-PAC PA200 HPLC column and a salt gradient at neutral or basic pH.

### BACKGROUND OF THE INVENTION

Significance: Protein-DNA binding plays a central role in gene regulation and by that in all processes in the living cell. Experimental and computational approaches facilitate better understanding of protein-DNA binding preferences via high-throuput measurement of protein binding to a large number of DNA sequences and inference of binding models from them (References 21-24 [0004]). To the best of our knowledge all current technologies, such as protein-binding microarrays, high-throuput SELEX, as well as the integrated microfluidic approach are indirect, expensive, and time-consuming methods testing the affinity of oligonucleotides instead of the actual dsDNA. In this application we claim the invention of a novel technology to directly assess the sequence, i.e. map the position within a specific dsDNA where a protein binds. We have developed a technology that labels dsDNA (see FIG. 1 for the chemical structure of the labeling agent), and based on well established physico-chemical principles, leaves the DNA regions bound to the protein intact. We wish to patent this ds nucleic acid-specific labeling technology (FIG. 2), as well as the approach of mapping protein-bound regions of dsDNA, using nanopore-based DNA sensing (FIG. 3).

Background: Osmium tetroxide 2,2′-bipyridine (OsBp) is a useful agent that reacts cleanly with pyrimidines (Reference 7 [0004]). It was proposed as a ssDNA contrast agent 60 years ago to facilitate electron microscope-based DNA sequencing (References 8-10 [0004]). In later years it was used extensively to probe DNA nicks, because it reacts freely with unpaired pyrimidines, but not with paired pyrimidines, as in dsDNA. We have recently developed methods for labeling efficiently ssDNA with OsBp to produce practically Thymidine (T)-labeled strands using Protocol A, or 100% all pyrimidine-labeled strands using Protocol B (References 1-3 and 5,6 [0003]). Protocol A and Protocol B modified ssDNA is being proposed as a surrogate for nanopore-based DNA sequencing and has shown promising results. This technology was recently filed as a non-provisional patent application [0002].

Innovation: To label dsDNA has proven challenging. Osmium tetroxide, 1,10-phenanthroline (Os,phen) was reported to label dsDNA, but severely deforms the ds structure (Reference 11 [0004]). Protocol B developed by us in the presence of denaturing agents, such as 7M Urea, results in labeling dsDNA, but at the same time denaturing it to yield two strands of osmylated ssDNA (DNA(OsBp)). It is critically important not to distort the ds nature of the DNA during labeling, because if this structure is distorted the protein will very likely dissociate and the “mapping” of its position on the dsDNA will be unsuccessful. Here we wish to report that we have developed a novel methodology, Protocol C, whereas OsBp labels primarily Ts in dsDNA without disrupting the ds structure. Following Protocol C, dsDNA is partially osmylated, ds structure is undistorted and hence protein-bound regions are protected and remain unmodified (FIG. 2). After quickly denaturing away the protein and removing the excess label, dsDNA can be probed to find the region(s) where the protein was bound. Suitable nanopores for ds DNA were shown to discriminate the presence/absence of the OsBp label (Reference 4 [0003]). In addition, detecting the unlabeled region could be also be accomplished by nanopore-based osmylated ssDNA sequencing after double strand denaturation (References 3 and 5 [0003]).

Traditional DNA Sequencing: The rapid, reliable, and cost-effective analysis and sequencing of nucleic acids is a major goal of government, researchers, and medical practitioners. Established DNA sequencing technologies have considerably improved in the past decade, but still require substantial amounts of DNA and several lengthy steps, while struggling to yield contiguous read-lengths of greater than 500 nucleotides. This information must then be assembled “shotgun” style, an effort that depends non-linearly on the size of the genome and on the length of the fragments from which the full genome is constructed; these steps are expensive, time-consuming and yield only a draft genome sequence.

Nanopore-based sequencing has been investigated for the last 20 years as an alternative to traditional sequencing approaches (References 14-17 [0004]). This method involves passing a nucleic acid, for example single stranded DNA (ssDNA), through a nanometer wide opening while monitoring a signal, such as an electrical signal, that is influenced by the physical properties of the nucleic acid subunits as the analyte passes through the nanopore opening. The nanopore optimally has, at least one section, of the appropriate size and the three-dimensional configuration that allows the analyte to pass in a sequential, single file order. Under theoretically optimal conditions, the polymer molecule passes through the nanopore at a rate such that the passage of each discreet subunit of the polymer can be correlated with the monitored signal. Differences in the chemical and physical properties of the subunits that make up the polymer, for example the nucleotides that compose the ssDNA, result in characteristic electrical signals. Nanopores, such as for example, protein nanopores held within lipid bilayer membranes (FIG. 3) and solid-state nanopores, which have been used for analysis of DNA and RNA, provide the potential advantage of robust analysis of polymers even at low copy number.

However challenges remain for the full realization of such benefits. For example, the five nucleotides (A, G, T, C, U) that are the canonical subunits of nucleic acids are chemically comparable and produce similar signals during translocation, therefore making their discrimination challenging. Additionally, nanopores are entities of definite length, and have recognition sites for a sequence of nucleobases, in contrast to recognition for a single base (Reference 16 [0004]). Hence the observed signal corresponds to a sequence and not a single base, making the correlation of the signal to bases ambiguous. All these issues create unacceptable error in “base-calling”. Another major issue with nucleic acid translocation via nanopores is that translocation per base is too fast to be resolved by contemporary state-of-the-art instruments. In order to address this problem, the field has instituted the use of enzymes, polymerases and others, which have the ability to move the nucleic acid one base at a time.

The above enzyme-motor development has been used with relative success, slowing down the translocation to easily detectable levels. Nevertheless such enzymes have proofreading functions and they do not always move the strand forward. Moreover the enzyme's movement is sometimes interrupted, which confuses the reading process, i.e. some parts of the nucleic acid are either read twice or not at all. Furthermore these enzymes are costly, and relatively slow in processing the strand. Specifically the enzymatic assistance results in translocation speeds that are 100 to 1000-fold slower compared to what current state-of-the art instruments can detect. The additional drawback of the enzymes is that they typically dissociate from the nucleic acid and sequencing is interrupted, yielding typical reading lengths that are less than 5000 nt. Hence the development of a sequencing technology that avoids enzymatic assistance is urgently needed.

Accordingly, a need remains to avoid the use of enzymes, a need to find another way to slow down the translocation of nucleic acids via nanopores, and also a need to clearly distinguish each nucleobase from the others. The methods and compositions of non-provisional U.S. patent application Ser. No. 14/944,888 (cited above under [0002]) address all three issues, and related needs of the art.

Nucleic acid Labeling agents: In the 1960s nucleic acids were reacted with metalorganic labels, used as contrast agents, and evaluated as substrates for obtaining sequencing information by electron microscopy (References 8-10 [0004]). Osmium tetroxide 2,2′-bipyridine (OsBp) was exploited as an agent to label the pyrimidines in both ssDNA and ssRNA, and monofunctional platinators were exploited as agents to label the purines. Frequently OsBp was also used to label unpaired Ts in dsDNA, followed by cyclic voltametry detection (Reference 7 [0004]). Cis-platin is a bifunctional platinator, known to react with adjacent Gs, but it has additional reactivity and forms crosslinks between strands, so it is not a useful label for sequencing purposes. The EM sequencing approach encountered a number of obstacles and did not yield tangible results.

Among the unresolved issues that prohibits investigators from pursuing the labeling nucleic acids approach (References 18-20 [0004]) for sequencing as well as for other applications are (i) efficient and homogeneous labeling has not been reported, and (ii) no validated analytical tool exists to check a labeled polymer base by base and determine false positives and false negatives. Most importantly it is known that ss nucleic acids have tertiary structure, and hence the conjecture is made that the tertiary structure prohibits homogeneous labeling. Homogeneous labeling is a critical attribute for any nucleic acid label intended to facilitate sequencing. If labeling does not occur homogeneously, i.e., independent of length, sequence and composition, then the number of false negatives would be large and unpredictable, leading to erroneous “base calling”.

In the non-provisional U.S. patent application Ser. No. 14/944,888 (cited above under [0002]) we describe methods that yield predictable and homogeneous labeling independent of nucleic acid length, sequence, composition, and tertiary structure, as well as analytical methods to determine and confirm the extent of labeling. We disclose and describe specific protocols that osmylate any nucleic acid to exactly the same extent, i.e., % T(OsBp) and % C(OsBp) without prior knowledge of sequence, length or composition even when this polymer has tertiary structure. We are now extending the technology of labeling ss nucleic acids to labeling ds nucleic acids. This extension could not have been predicted based on scientific principles, and earlier literature advises otherwise (References 7, 11 [0004]). The specific and substantial utility to label ds nucleic acid in a predictable way, use of the disclosed assays to confirm labeling, as well as preserve the double stranded nature of the polymer may have several uses including protein mapping. This novel technology, abbreviated Protocol C, will be described in the “Detailed description of the invention” section.

### BRIEF SUMMARY OF THE INVENTION

This invention combines two different fields of science, i.e. osmylated nucleic acids and nanopore-based characterization and/or sequencing, in a novel way that is utilized, among others, for fast, high-throuput, and inexpensive protein mapping on dsDNA. We claim invention relating to the methods to label ds nucleic acids without distorting the double helical configuration, thus preserving the bound protein. We claim that this labeling can be done predictably, and we also claim the analytical methods available to monitor this transformation/labeling. Further more we exploit well established protein removal biochemical methods to remove the protein. Then we use the disclosed purification method to remove excess label and yield practically 100% of the ds nucleic acid. Furthermore we describe here a simple quality control assay in order to confirm extent of labeling. We also claim invention as it pertains to utilizing our prepared partially labeled dsDNA, and nanopore measurements via nanopores suitable for dsDNA (or suitable for the resulting ssDNA) to yield characterization of the labeled dsDNA in the presence/absence of the bound protein and from the differences map the location where the protein binds.

Experiments conducted by the inventor and described here illustrate proof-of-principle that dsOligos, and dsDNA, lambda is used here as an example of dsDNA, are partially labeled without ds structure distortion. In addition, proof-of-principle is obtained from experiments at a collaborator, Professor Meni Wanunu, at Northeastern University in Boston using solid-state nanopores with a 202 bp long dsOligol [SEQ ID NO:3], labeled by this inventor, of successful translocation via a suitable solid-state nanopore. The data exhibit strong discrimination between the osmylated 202 bp and the intact material (see FIG. 4, [SEQ ID NO:3]). Therefore the invention has been validated in, at least, one nanopore platform, and osmylated dsDNA, using the methods disclosed in this invention presents a novel and substantial utility in several applications including protein mapping. In the section on “Detailed description of the invention” we include all the evidence that led to this invention.

In all embodiments the pyrimidine-specific label is osmium tetroxide 2,2′-bipyridine (OsBp).

In some embodiments the nucleic acid is a ds oligodeoxynucleotide (dsOligo).

In some embodiments the nucleic acid is ds lambda.

In some embodiments the labeled polymer is partially osmylated.

In some embodiments the labeled polymer is fully osmylated.

In some embodiments the nanopore is solid-state (SiN) with 4.4 nm wide pore and the dsOligo is a 202 bp where a relatively high percentage of Ts are in the form of dT(OsBp) interspersed among intact nucleotides.

### DESCRIPTION OF DRAWINGS

FIG. 1 illustrates the reaction between Osmium tetroxide and 2,2′-bipyridine that forms a complex with a small equilibrium constant. This complex (bipy-OsO4, or Osbipy, or OsBp) reacts in a next step with a pyrimidine (deoxythymidine monophosphate shown) by addition to the C5-C6 double bond of the pyrimidine ring to form a conjugate. A similar product is formed by addition to the C5-C6 double bond of cytidine or uracil. The reaction is independent on the presence of ribose or deoxyribose as well as independent of whether the reactant is a nucleoside or a nucleotide, mono-, di-, or triphosphate, or a unit within a polymer. The actual conjugate is a topoisomer formed by addition from the top or the bottom of the pyrimidine ring. Hence the products are two isomers that are resolved by capillary electrophoresis (CE) or High performance liquid chromatography (HPLC), in the case of short oligos. In principle, the OsBp moiety does not interfere or prohibit base pairing. One way to illustrate the difference between osmylated and intact bases is to compare (molecular weight) of each: dC (111), dT (126), dA (135), dG (151); dC-OsBp (521), dT-OsBp (536), i.e. osmylation adds about 400% mass to the reactive base compared to an unreactive one.

FIG. 2 illustrates the strategy for protein mapping using this technology. Protocol C, whereas OsBp labels mostly Ts in dsDNA without disrupting the ds structure. Following Protocol C, dsDNA is partially osmylated at no more than 50% of the pyrimidines and most likely to a great extent T-osmylated. However protein-bound regions are protected and remain unmodified (see FIG. 2). After denaturing away the protein, dsDNA can be probed to find the region(s) where the protein was bound. Suitable methods to do this mapping will discriminate the presence/absence of the OsBp label. Detecting the unlabeled region can be done by methods suitable for dsDNA (Reference 4 [0003]), or for ssDNA after denaturation. Nanopore-based osmylated ssDNA sequencing is an example of such method [0002].

FIG. 3 illustrates a representation of the translocation of ssDNA via the a-Hemolysin nanopore (α-HL) showing the 1.4 nm constriction zone and the rather long but confined b-barrel; voltage (positive, trans to cis) across the insulated nanopore leads to ion current via the pore and threading of the ssDNA, which obstructs the current when inside the pore. Solid state nanopores are even simpler in construction as they do not include a protein and no lipid bilayer either. Still the actual principle for characterization of nucleic acids is the same for solid-state nanopores as described for alpha-Hemolysin above.

FIG. 4 illustrates detection of partially osmylated dsDNA by a solid-state nanopore (SiN, diameter 4.4 nm). Control is 202 bp and R1 refers to the partially osmylated 202 bp [SEQ ID NO:3]. Open pore current at about 1.75 nA with about 20% current reduction during translocation of the intact dsOligol [SEQ ID NO:3] and about 50% current reduction during translocation of the partially osmylated dsOligol [SEQ ID NO:3]. This figure shows concatenated events with 20,000 baseline data points separating each event. Data is shown after low pass filtering at 200 kHz. Taken from Reference 4 [0003].

TABLE 1 lists nucleic acids used for the experiments illustrated in later Figures. Listed are the abbreviations, sequences, and the SEQ ID NO (see Sequence Listing).

TABLE 2 lists the experiments performed with a series of nucleic acids and a specific OsBp stock solution (abbreviated OsBpi). The table lists the percent of OsBpl in the reaction mixture, the time of incubation and the areas of the peaks observed in the CE profiles. The measure R(312/272) directly shows extent of osmylation. Published data confirm that R(312/272)=2.01×{fraction of labeled pyrimidines}/Nₜₒₜₐₗ ([0002] and References 2 and 5 [0003]). Considering that in natural DNA the fraction of pyrimidines is about equal to the fraction of purines, then a fully labeled DNA, i.e. all pyrimidines osmylated, will exhibit R(312/272)≈1.0, valid for both ss and dsDNA. All the experiments we have conducted illustrate that when R(312/272)>0.5, then a large portion of the nucleic acid becomes denatured.

TABLE 3 lists the experiments performed with a series of nucleic acids and a specific OsBp stock solution (abbreviated OsBp2). The table lists the percent of OsBp2 in the reaction mixture, the time of incubation and the areas of the peaks observed in the CE profiles. For information on the measure R(312/272), please read the description of Table 2.

TABLE 4 lists two experiments performed with a dsOligo2 (hybrid of SEQ ID NO:4 and of SEQ ID NO:5) prepared in situ by hybridization of the two complements, purchased as separate entities. Labeling was conducted using OsBp2 at two different concentrations to show that higher concentration is speeding up the reaction, only that the effect is not linear.

FIG. 5 is a plot of the kinetic data of the 5^{th} experiment with dsLambda in the presence of 60% OsBp2 from Table 3 assessed as a pseudo-first order reaction where the slope of the line provides the rate of the reaction at the specific conditions as 0.0226 per min, or a half-life of 31 minutes.

FIG. 6 is a plot of the kinetic data of the 1st experiment from Table 4 with dsOligo2 (hybrid of SEQ ID NO:4 and SEQ ID NO:5) assessed as a pseudo-first order reaction with a second order polynomial fit. The first-order slope of the line provides the rate of the reaction at the specific conditions as 0.0305 per min, or a half-life of 23 minutes.

FIG. 7 is a plot of the kinetic data of the 2^{nd} experiment from Table 4 with dsOligo2 (hybrid of SEQ ID NO:4 and SEQ ID NO:5) assessed as a pseudo-first order reaction with a second order polynomial fit. The first order slope of the line provides the rate of the reaction at the specific conditions as 0.0142 per min, or a half-life of 49 minutes. FIGS. 5 to 7 underscores that labeling of dsDNA is slower compared to labeling of dsOligos, most likely due to the stronger association as a consequence of the length of the double strand.

FIG. 8 illustrates the CE profile of intact dsLambda that appears as a number of peaks most likely due to inhomogeneity. It should be noted that dsDNA typically appear as very sharp peaks that migrate late, in contrast to ssDNA that appear as broader cone-shaped peaks that migrate several minutes earlier on the same CE method.

FIG. 9 illustrates the CE profile of partially osmylated dsLambda that appears as multiple peaks due to the fact that osmylation occurs randomly and therefore all types of partially osmylated products are plausible.

FIG. 10 illustrates the CE profile of a fully osmylated Lambda that is primarily single stranded, as evidenced by the very early migration time as well as the broad shape of the main peak.

### DETAILED DESCRIPTION OF THE INVENTION

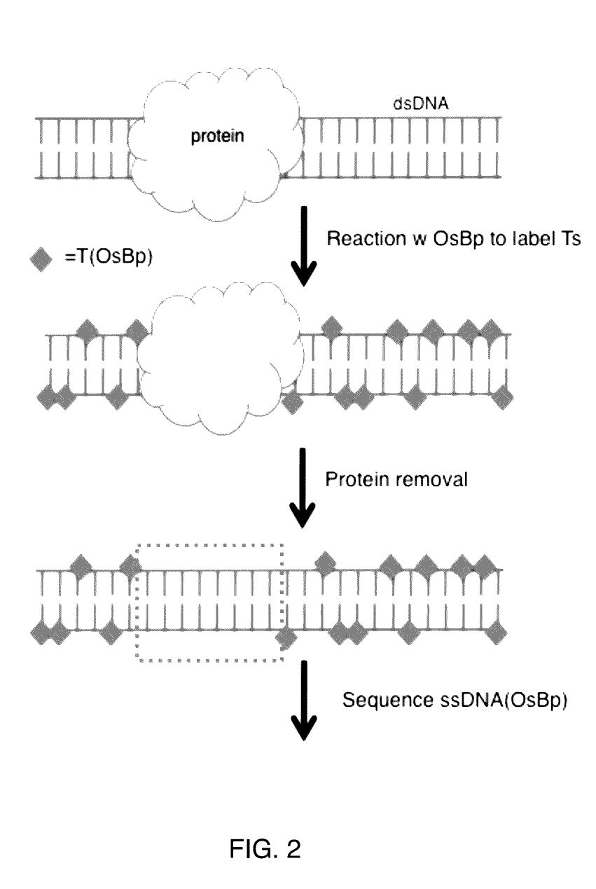

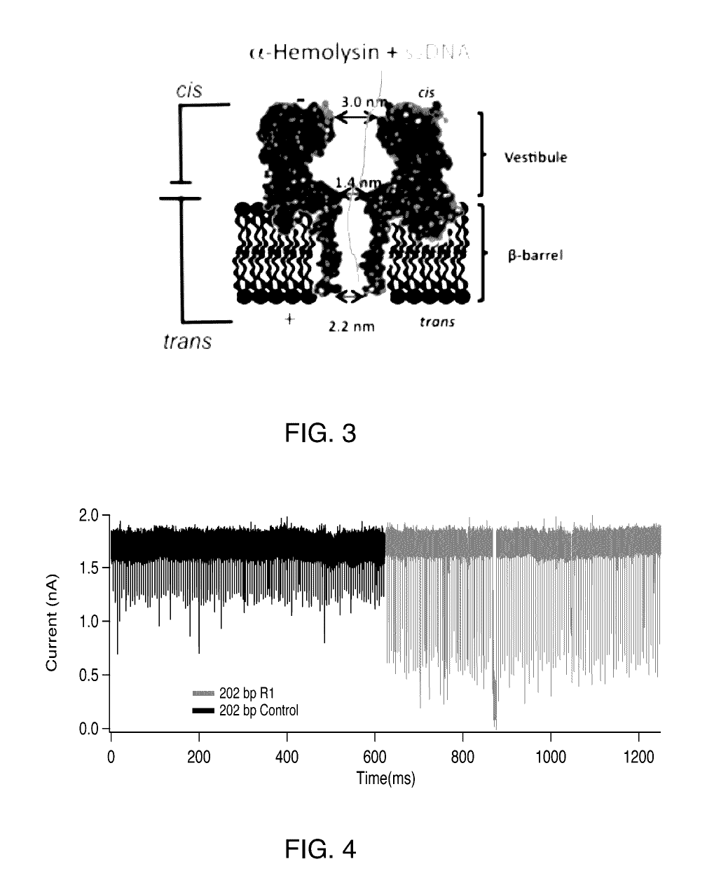

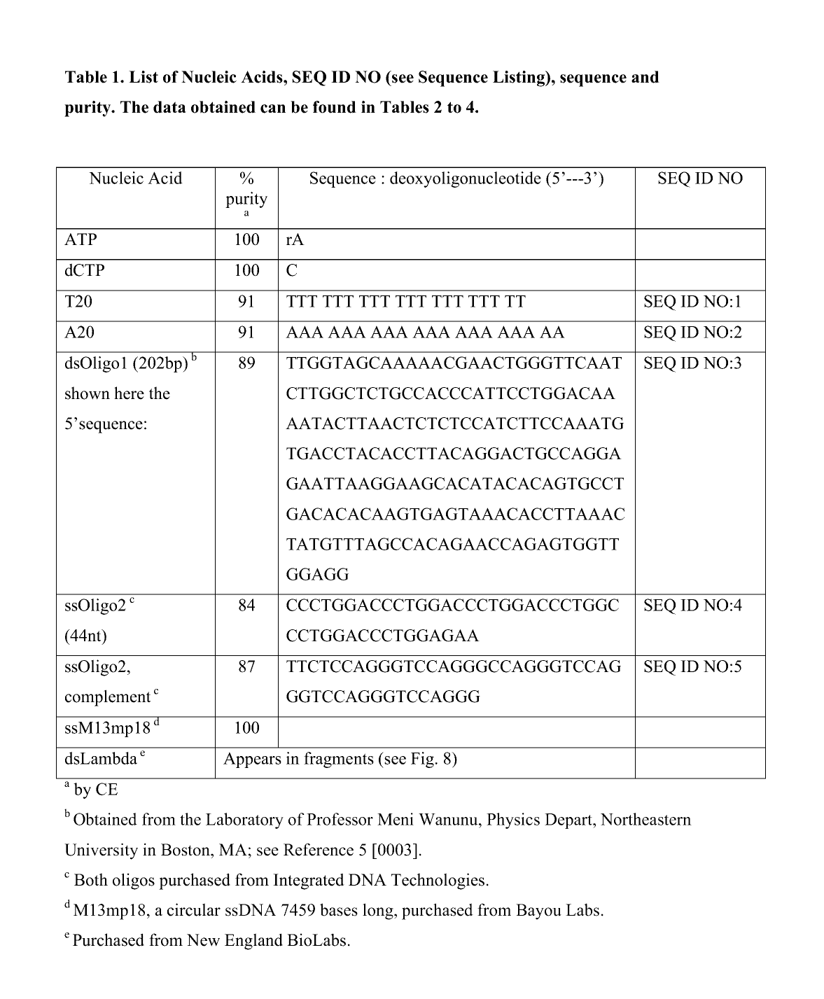

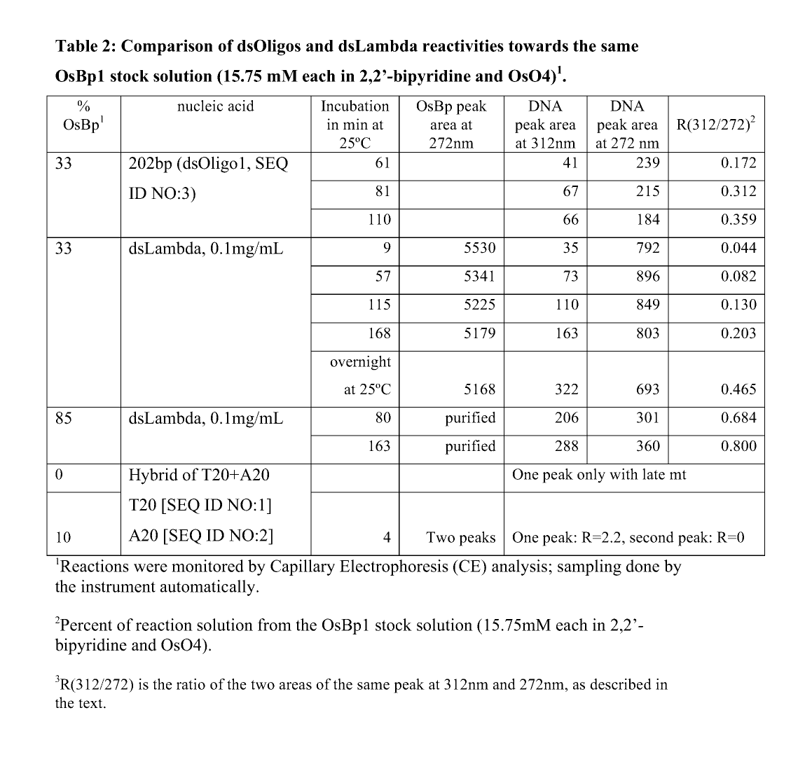

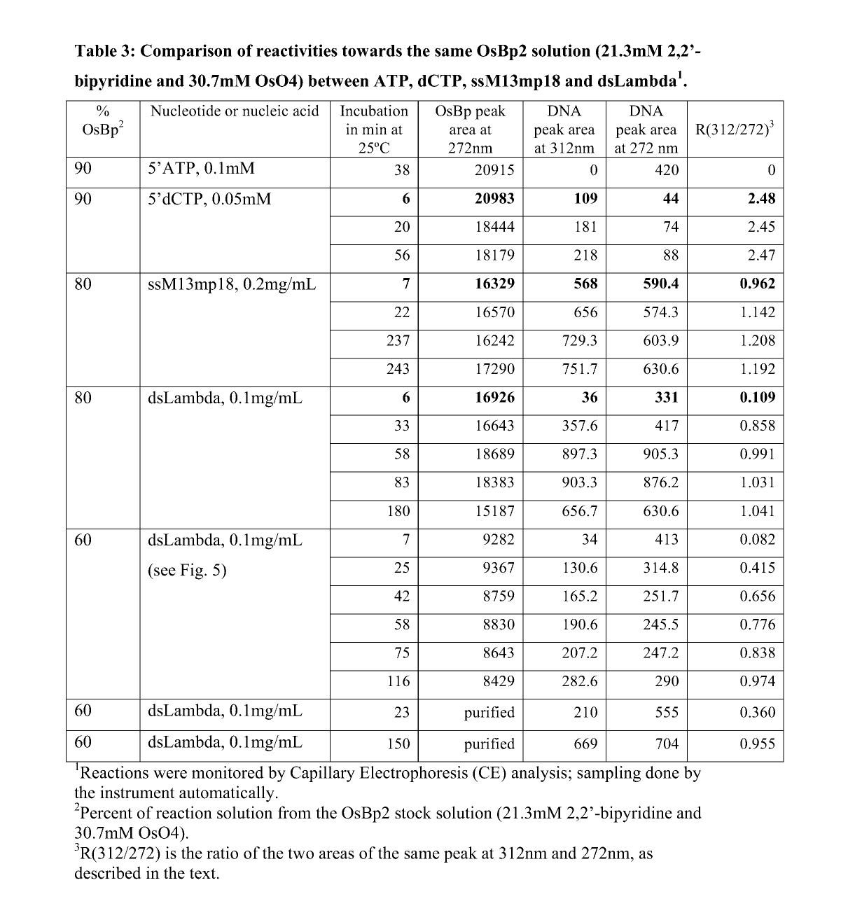

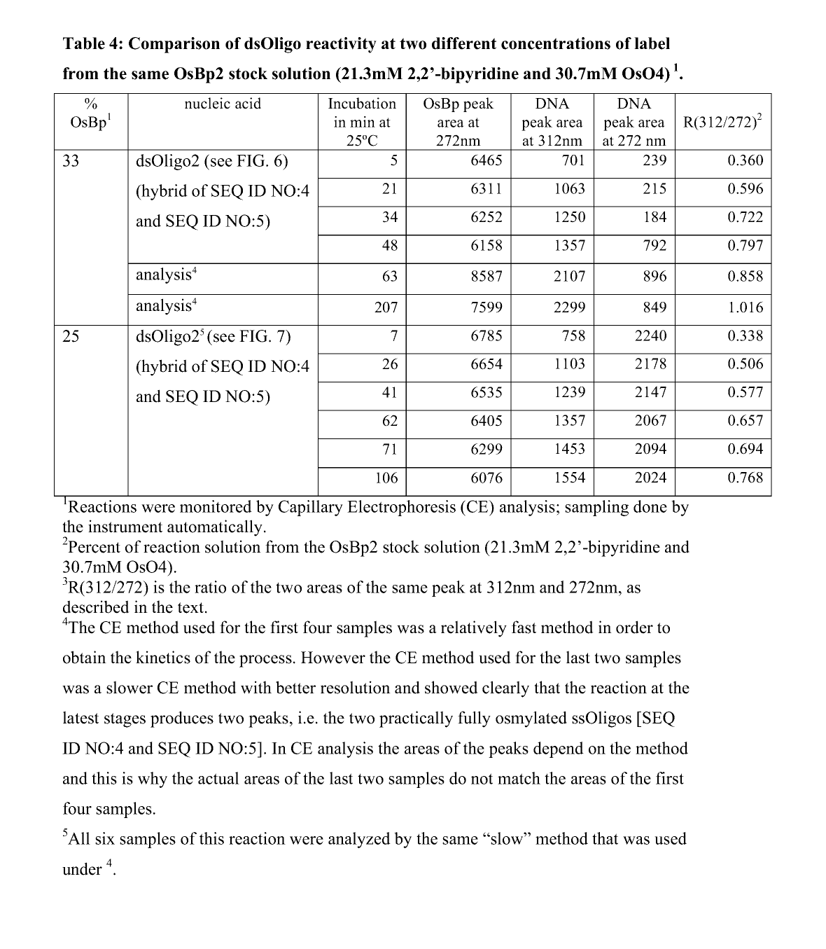

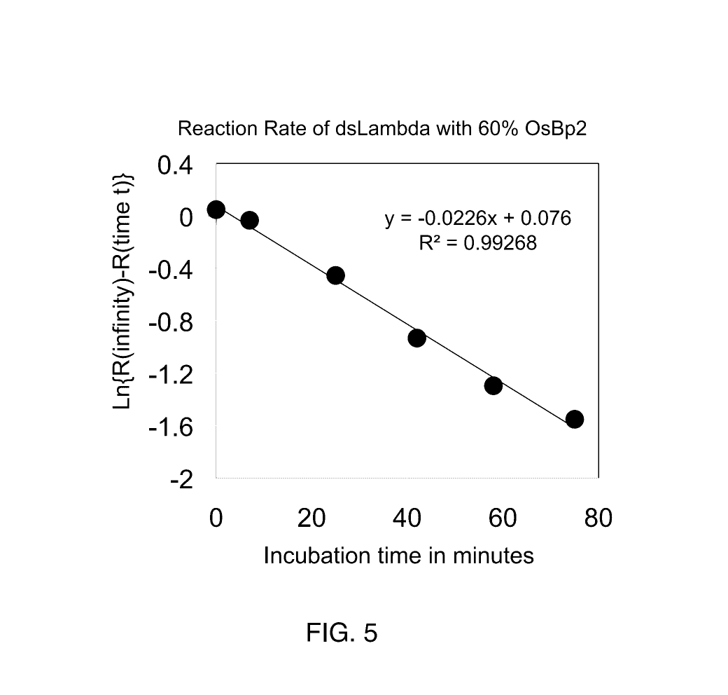

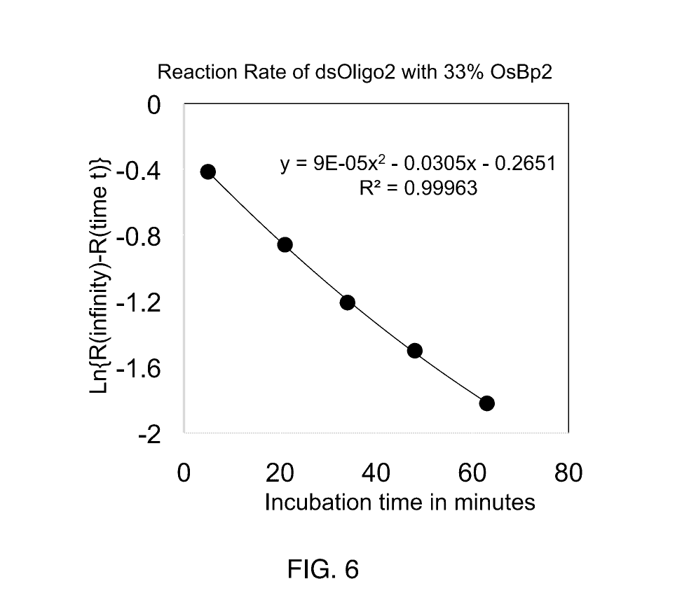

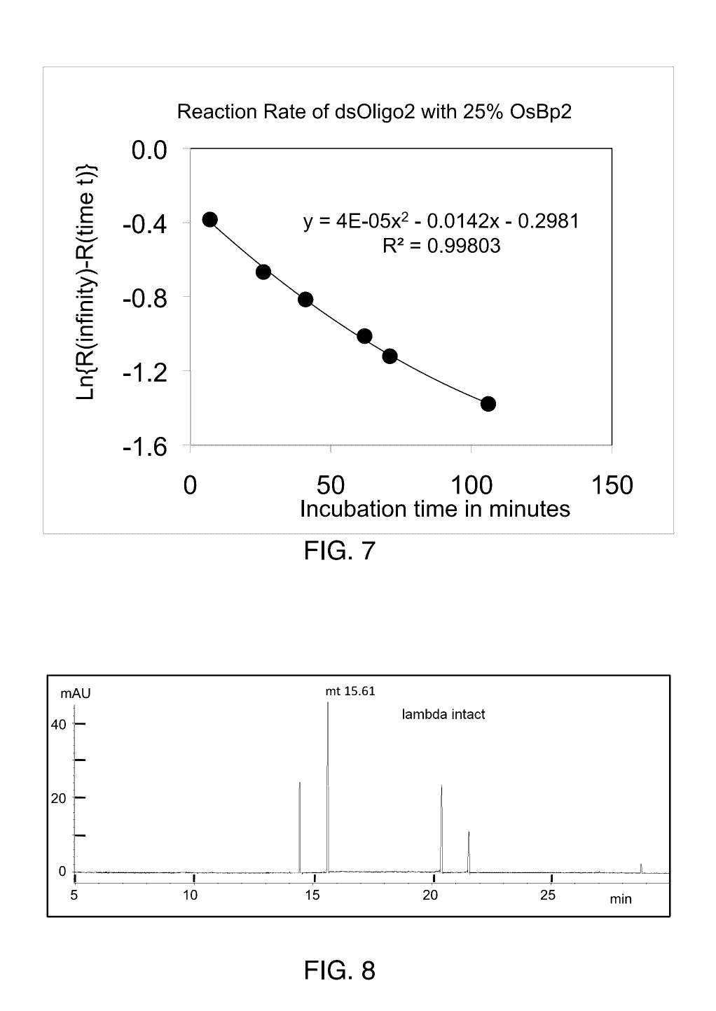

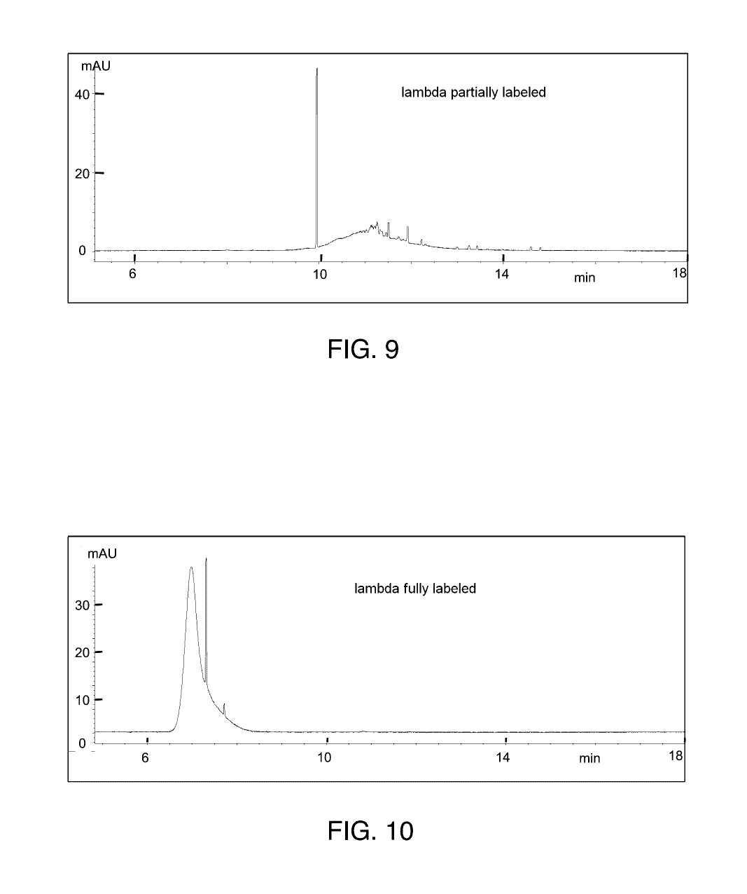

Summary: The present invention claims that self-structured nucleic acids, including dsDNA, may be partially osmylated while keeping the double stranded structure intact. Extent of labeling is predictable that can be monitored by well established analytical techniques, such as HPLC and CE (methods disclosed here). Extent of labeling is confirmed, via the UV-vis assay, after purification from the excess label. Both purification and UV-vis assay are disclosed here by the inventor. This invention may have several applications including mapping proteins bound on self-structured or double stranded nucleic acids, as follows: The bound protein protects the region of the nucleic acid at the location it binds and, based on well established physico-chemical principles that region remains intact, namely pyrimidines in that region do not become labeled (FIG. 2). Since the disclosed conditions of labeling do not distort the ds structure, the protein remains bound on the DNA and protects the specific region during osmylation. In order to quickly determine the location of the bound protein, among other biochemical methods, we here disclose nanopore-based characterization/sequencing. Such characterization is enabled by the presence of the osmylated pyrimidines that slow down translocation via suitable nanopores (see FIG. 4). We also disclose here proof-of-principle that both natural and solid-state nanopores easily discriminate between intact and labeled bases (see FIG. 4). Hence osmylated nucleic acids enable unassisted, nanopore-based characterization/sequencing with no limit in the length of the polynucleotide due to its enzyme-free implementation. Comparison of the current vs. time (i-t) traces from the nanopore translocations of the osmylated dsDNA in the presence/absence of the bound protein will pinpoint the region of the dsDNA where the protein was bound.

Osmylation of pyrimidines: Earlier publications of others used Osmium tetroxide and amines at various experimental conditions to label pyrimidines. For a review see Reference 7 [0004]. A detailed description of this inventor's development work to label pyrimidines with OsBp is provided in [0002]. Furthermore several publications describing numerous results solidifying this technology have appeared the last few years in peer-reviewed journals (see References 1-6 [0003]).

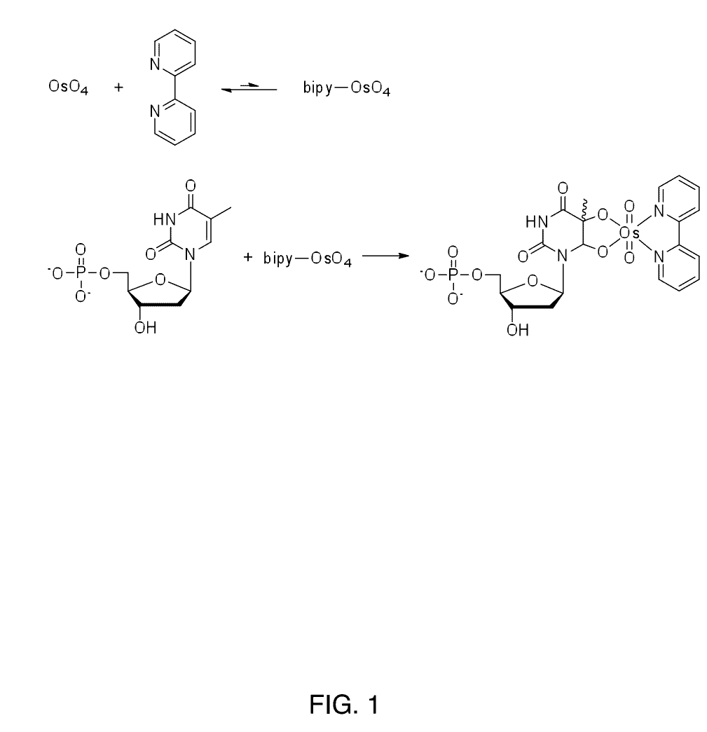

Preparation of OsBp stock solutions: In one embodiment the inventor prepared a 1:1 molar mixture of Osmium tetroxide (4% aqueous solution purchased from Electron Microscopy Sciences) and 2,2′-bipyridine (99+ purity purchased from Acros Organics) in glass vials in water at a final concentration of 15.75 mM each (OsBp1 stock solution, see FIG. 1). In another embodiment a stock solution of OsBp2 was prepared at about 30 mM OsO4 and 21 mM 2,2′-bipyridine. Please note that 21 mM 2,2′-bipyridine is very close to its solubility in water and this solubility is not affected by the presence of OsO4. Nevertheless the reactivity of OsBp2 is higher compared to the reactivity of OsBp1 primarily due to doubling the concentration of OsO4. Stock solutions at higher concentration than 15.75 mM are more suitable in labeling self-structured nucleic acids, as it will be elaborated below. There is no limit in the different OsBp stock solutions that one can prepare and we claim this generalization, as well as describe the methods to use them below. It should be mentioned that OsO₄ is volatile and dangerous. Safety precautions must be taken when preparing the stocksolution and the reaction mixtures. Because the equilibrium constant is relatively small, most of the OsO₄ in the OsBp solution is also in free form, so the OsBp solutions and mixtures with oligos are equally dangerous. Nevertheless the concentration is in the mM range and if reactions are carried under a hood, the danger is minimized.

Labeling procedure: Stock solutions of OsBp prepared as described above were mixed with ds lambda or dsOligos in water at different initial concentrations at room temperature and allowed to react, while the reaction mixture was monitored automatically by CE (see Tables 2-4). The reaction was always conducted in a glass vial, in water, with no buffer added. Most buffers react with OsBp and lower its concentration and pH control was found to be unnecessary. Reaction mixtures need to be placed in glass vials, because other materials react with OsBp and lower its effective concentration yielding irreproducible results. Labeling is evidenced by a shift in the migration time of the polymer towards earlier times and by an increase in absorbance at 312 nm (unlabeled DNA has less than 0.02 AU at 312 nm. Please note that this absorbance is not limited to 312 nm but is valid for the range 300 nm to 330 nm). It is critically important that pseudo-first order conditions are applied and therefore the concentration of the label in the reaction mixture should be 20 to 30-fold higher compared to the pyrimidine concentration measured in monomer units. However the actual concentration of the oligo does not influence the rate of reaction as shown in [0002]. Also critically important is that the OsBp concentration is high enough so that the labeling reaction is complete in no more than couple of hours, because we have evidence that prolonged incubation has a deterrent effect on the double stranded structure (see Table 2, 2^{nd} experiment). In addition the concentration of OsBp should not be very high and lead to within minutes labeling of the dsDNA, because in this case the quenching of the reaction can not be properly controlled, and the reaction will run off to produce labeled ssDNA which is undesirable for the protein mapping application.

Recommended labeling conditions (Protocol C): Other conditions, besides Protocol C, are also usable and can be deduced from the experiments listed in Tables 2-4. Protocol C: 10 to 20 uM dsDNA in 60% OsBp (about 30mM 0s04/20mM 2,2′-bipyridine) incubated for 30 min in water at 25±2 ° C. yields 35 to 50% labeling and mostly undistorted ds structure. Removal of the label, immediately after the 30 min incubation, is necessary in order for the reaction to stop at that level of labeling. We found that higher level of labeling results in increasing amounts of ssDNA.

Novelty of technology #1: In contrast to a published report from Chang, Beer, and Marzilli (Reference 10, see page 37, 1^{st} paragraph) who were unable to find conditions to selectively osmylate T over C, the current inventor discovered such conditions and discloses them in [0002].

Novelty of technology #2: In contrast to published results from Nomura and Okamoto (Reference 12), invention [0002] recommends conditions that lead to comparable reactivity of Ts independent of composition. The comparable reactivity is important because it leads to one protocol for T-osmylation for any nucleic acid. It is likely that this comparable reactivity of the Ts is also valid in ds DNA and results in a random partial labeling of the whole ds molecule with the exception of the region where the protein is bound.

Novelty of technology #3: The conclusion for undetectable osmylation of G (see Reference 6 [0003]) is in contrast to observations published elsewhere (Reference 13 [0004]), and it is attributed to the different conditions used in the two studies. Table 3, 1^{st} experiment, also shows that both A, as well as the ribo component are completely unreactive towards the highest concentration of OsBp2.

Evidence that double stranded structure is preserved: There are two lines of evidence that strongly suggest the practically complete preservation of the double stranded nature of the polymer. The first line of evidence relies on the fact that the migration time of dsDNA or dsOligo on CE is many minutes later compared to the migration time of ssDNA or ssOligo. Increasing extent of labeling yields to earlier migration times, but the order does not change. Hence osmylated dsDNA appears much later compared to osmylated ssDNA. The second line of evidence relies on the actual kinetics of labeling. Depending on the OsBp concentration, labeling dsDNA is 10- to 20-times slower compared to labeling of ssDNA. For example, Table 3 illustrates that 5′dCTP is 100% labeled within 6 minutes (R(312/272)=2.48, infinity value) and under slightly lower OsBp concentration ssM13mp18 is also practically fully labeled in 7 minutes(observed R=0.962 compared to infinity R=1.19), just as claimed in [0002]. However at the same conditions where ssM13mp18 is practically labeled, dsLambda is only about 11% labeled. This kinetic comparison strongly suggest that one is looking at two different processes: One process is labeling of ssDNA and the other is labeling dsDNA.

Our observations are consistent with practically 100% of pyrimidines labeling of dsDNA yields labeled ssDNA (FIG. 10) and this is why we recommend that for protein mapping labeling of dsDNA remains below 50%. Since we expect that the selectivity of T osmylation in favor of C osmylation shown for ssDNA may still be applicable to dsDNA, 50% of labeling implies that it is primarily T that is labeled and C remains practically unlabeled. It is due to the strong association of the C-G base-pairing that prevents denaturation, and once C is labeled the ds structure is denatured. The last experiment in Table 2 of a 20-bp long AT double helix, that is not being preserved under the influence of relatively weak osmylation conditions, strongly confirms the importance of the C-G base pairing.

Purification methods to remove small molecules from polymers are many (look up nucleic acid purification kits) and we validated one of them, namely the spin columns TC FC-100 from TrimGen. One or two passes are sufficient to remove up to 30 mM of OsBp, with excellent recovery of the labeled polymer, including dsLambda.

Quality control UV-Vis assay: The infinities (constant R(312/272) of the various experiments listed in Tables 2 to 4, provide strong evidence that the measure of the extent of osmylation (R(312/272)), discovered and disclosed in [0002] is also valid for dsDNA. The equation R(312/272)=2.01×(T+C)/Nₜₒₜₐₗ can be used to assess extent of osmylation with dsDNA. Evidently, one can use the assay to determine extent of osmylation, even if one does not use the recommended protocol, because this assay is based on the thermodynamic property of the osmylated polymer.

Rate determination of a process provides detailed mechanistic insights into a reaction and allows for predictability. For detailed information on the rate determinations from the timed values of R(312/272) included here in FIGS. 5 to 7, References 1 and 2, as well as [0002] are valuable resources.

Stability of osmylated polymers: All our observations are consistent with high room temperature stability of the osmylated polymers in the aqueous solutions. Still one is advised to store them at −20C. Osmylated-C is shown to suffer 1 to 2% degradation per hour at room temperature (Reference 6 {0003]). However since osmylated dsDNA is anticipated to have very little osmylated-C, there appears to be no venue for its degradation.

In conclusion, these data demonstrate that double stranded osmylated nucleic acids can be prepared easily, predictably, and accurately characterized using R(312/272). They have specific and substantial utility including nanopore-based profiling applications. While embodiments have been illustrated and described, it will be appreciated that various changes can be made therein without departing from the spirit and scope of the invention.

What is claimed is:

1. Methods for labeling double stranded nucleic acids (referred to here as osmylated or labeled ds polymers) in the presence/absence of a bound protein comprising:
Using Osmium tetroxide 2,2′-bipyridine of a recommended preparation at recommended conditions in order to partially label the pyrimidines of a double stranded nucleic acid without distoring the double helical structure and consequently without denaturing/removing any bound protein. Once the recommended extent of labeling is achieved quickly removing the protein by standard biochemical procedures; purifying the product by one or more purification methods to remove the unreacted label; and using one or more analytical methods to characterize the article and confirm extent of labeling by the disclosed assay.

2. A method of determining the position of the bound protein on the double stranded polymer, by comparing the double stranded polymer after labeling it under identical conditions but in the presence/absence of the protein comprising:
applying an electric field across a nanopore disposed between a first conductive liquid medium and a second conductive liquid medium and
measuring the ion current for both labeled polymers (with or without bound protein) and assessing the region in the sequence where the two polymers (with or without bound protein) differ.

3. A kit for performing the method of claim 1, comprising, in separate compartments,
a) the label,
b) the removal of the protein component
c) the purification component to remove extra label,
d) instructions for using a), b), and c) in series, and
e) instructions to do quality control test after performing c).
